(19) United States
(12) Patent Application Publication
(10) Pub. No.: US 2024/0386732 A1
(43) Pub. Date: Nov. 21, 2024

(54) ALGAE INFORMATION MANAGEMENT APPARATUS, NON-TRANSITORY COMPUTER-READABLE STORAGE MEDIUM, AND ALGAE INFORMATION MANAGEMENT METHOD

(71) Applicant: HONDA MOTOR CO., LTD., Tokyo (JP)

(72) Inventor: Nozomi SHIOBARA, Saitama (JP)

(21) Appl. No.: 18/632,317

(22) Filed: Apr. 11, 2024

(30) Foreign Application Priority Data
May 17, 2023 (CN) 202310556805.3

(51) Int. Cl. G06V 20/69; G06V 10/56

(52) U.S. Cl. CPC G06V 20/698; G06V 10/56

(57) ABSTRACT

An algae information management apparatus according to embodiments includes an inputter, a specifying unit, and a storage. The input unit receives input of first identification information given for each measurement microalgae. The specifying unit specifies second identification information given to a color sample closest to a color of the microalgae among a plurality of color samples for comparison with the color of the microalgae. The storage stores the first identification information and the second identification information in association with each other.

This application is based on and claims the benefit of priority from Chinese Patent Application No. CN202310556805.3, filed on 17 May 2023, the content of which is incorporated herein by reference.

### BACKGROUND OF THE INVENTION

### Field of the Invention

The present invention relates to an algae information management apparatus, an non-transitory computer-readable storage medium, and an algae information management method.

### Related Art

The constituents of algae vary depending on the environment in which the algae is grown. As the environment varies depending on the region, the algae changes in constituents depending on the region grown.
Patent Document 1: Japanese Unexamined Patent Application, Publication No. 2017-502680

### SUMMARY OF THE INVENTION

Algae have suitable applications depending on protein content. The constituents of algae vary depending on the environment grown as described above. For this reason, it is necessary to measure the protein content of the algae in order to utilize the algae for a suitable application.

Embodiments of the present invention each provide an algae information management apparatus, an non-transitory computer-readable storage medium, and an algae information management method capable of reducing the time and effort of measuring the protein content of a microalgae.

An algae information management apparatus according to embodiments includes an inputter, a specifying unit, and a storage. The inputter receives input of first identification information given for each measurement microalgae. The specifying unit specifies second identification information given to a color sample closest to a color of the microalgae among a plurality of color samples for comparison with the color of the microalgae. The storage stores the first identification information and the second identification information to be associated with each other.

According to embodiments of the present invention, it is possible to reduce the time and effort of measuring the protein content of a microalgae.

### BRIEF DESCRIPTION OF THE DRAWINGS

FIG. 1 is a block diagram showing an example of a management system and a main configuration of components included in the management system according to a first embodiment and a second embodiment;

FIG. 2 is a flowchart showing an example of processing by a processor in FIG. 1;

FIG. 3 is a view of an example of a screen displayed on a display device in FIG. 1; and

FIG. 4 is a flowchart showing an example of processing by the processor in FIG. 1.

### DETAILED DESCRIPTION OF THE INVENTION

Hereinafter, a management system according to some embodiments will be described with reference to the drawings. In the drawings used in the following description of the embodiments, the scale of each part may be appropriately changed. In addition, in the drawings used in the description of the following embodiments, the configuration may be omitted for the sake of explanation. In the drawings and the specification, like reference numerals denote like elements.

### First Embodiment

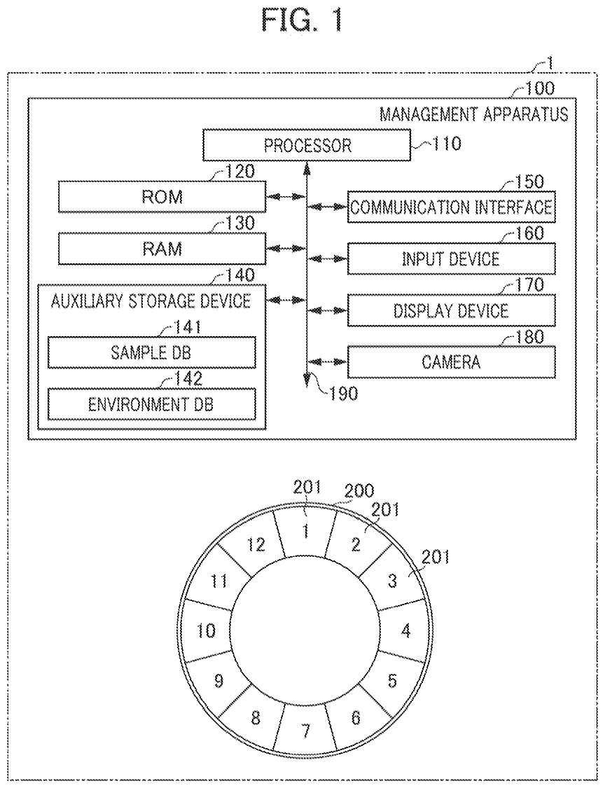

FIG. 1 is a block diagram showing an example of a management system 1 and a main configuration of components included in the management system 1 according to the first embodiment. Note that the external appearance of the color chart 200 is shown. Each component of each device may be built-in or external. The management system 1 is a system that stores and manages evaluation results and classification results of algae. The algae used in the management system 1 is grown using, for example, a culturing apparatus for culturing the algae. The algae used in the management system 1 is, for example, an algae dried product obtained by drying the algae. The algae used in the management system 1 is microalgae. The microalgae used in the management system 1 is preferably microalgae belonging to Chlamydomonas reinhardtii. This is because, with a microalgae belonging to Chlamydomonas reinhardtii, it is possible to easily identify a protein content from the color of the microalgae. The microalgae used in the management system 1 is more preferably Honda (Registered Trademark) DREAMO strain (accession number: FERM BP-22306). This is because, with the Honda DREAMO strain, it is possible to easily identify the protein content from the color of the microalgae.

The evaluation result managed by the management system 1 indicates the result of protein evaluation for measuring the protein content of algae. The classification result managed by the management system 1 indicates the result of application classification processing for classifying algae according to the protein content.

The management system 1 includes, for example, a management apparatus 100 and a color chart 200. The management system 1 may include some of them.

The management apparatus 100 is an apparatus that stores and manages evaluation results and classification results of algae. The management apparatus 100 is a general-purpose apparatus such as a personal computer (PC), a smartphone, or a server apparatus. Alternatively, the management apparatus 100 may be an apparatus dedicated to the management system 1. The management apparatus 100 includes, for example, a processor 110, read-only memory (ROM) 120, random-access memory (RAM) 130, an auxiliary storage device 140, a communication interface 150, an input device 160, a display device 170, and a camera 180. A bus 190 or the like connects these components. The management apparatus 100 is an example of an algae information management apparatus.

The processor 110 is a central part of a computer that performs processing such as calculation and control necessary for the operation of the management apparatus 100, and performs various kinds of calculations and processing. The processor 110 may be, for example, a CPU (central processing unit), a MPU (micro processing unit), a SoC (system on a chip), a DSP (digital signal processor), a GPU (graphics processing unit), an ASIC (application specific integrated circuit), a PLD (programmable logic device) or a FPGA (field-programmable gate array). Alternatively, the processor 110 may be a combination of a plurality of these. Further, the processor 110 may be made by combining a hardware accelerator and the like with these. The processor 110 controls each component to realize various functions of the management apparatus 100 based on programs such as firmware, system software, and application software stored in the ROM 120 or the auxiliary storage device 140. Further, the processor 110 executes processing described later based on the program. A part or all of the program may be incorporated in a circuit of the processor 110.

The ROM 120 and the RAM 130 are main storage devices of a computer having the processor 110 as a central element. The ROM 120 is non-volatile memory used exclusively for reading data. The ROM 120 stores, for example, firmware among the above programs. The ROM 120 also stores data used for the processor 110 to perform various kinds of processing.

The RAM 130 is memory used for reading and writing data. The RAM 130 is used as a work area or the like for storing data temporarily used when the processor 110 performs various kinds of processing. The RAM 130 is typically volatile memory.

The auxiliary storage device 140 is a computer auxiliary storage device having the processor 110 as a central component. The auxiliary storage device 140 is, for example, EEPROM (electric erasable programmable read-only memory), HDD (hard disk drive), flash memory, or the like. The auxiliary storage device 140 stores, for example, system software and application software among the above programs. The auxiliary storage device 140 stores data to be used when the processor 110 performs various kinds of processing, data generated by processing in the processor 110, and various kinds of setting values.

The auxiliary storage device 140 stores, as the data, for example, a sample DB (database) 141, an environment DB 142, color sample information, and classification information.

The sample DB 141 is a database that stores and manages information relating to algae measurement samples. The sample DB 141 stores sample IDs, growth IDs, sample evaluation information, sample classification information, and measurement date/time information in association with each algae measurement sample. The sample ID is unique identification information for each measurement sample. The growth ID is unique identification information for each algae growing environment. The growth ID associated with the sample ID indicates that the growing environment of the measurement sample specified by the sample ID is the growing environment specified by the growth ID. The sample evaluation information is information indicating the evaluation result of the protein evaluation of the measurement sample specified by the sample ID. The sample evaluation information includes sample color information and sample protein information. The sample color information is information indicating a color of the measurement sample. The sample protein information is information indicating the protein content of the measurement sample. The sample classification information is information indicating the result of the application classification processing of the measurement sample specified by the sample ID.

The environment DB 142 is a database that stores and manages information relating to the growing environment in which algae is grown. For example, the environment DB 142 stores the growing information, the environment evaluation information, and the environment classification information to be associated with the growth ID for each growing environment. The growth ID is unique identification information for each growing environment. The growing information is information indicating a growing environment. The growing information includes, for example, information indicating a growing region. Further, the growing information may include information indicating the growing season. The growth information may also include temperature, humidity, sunshine hours, or other weather related information. The environment evaluation information associated with the growth ID is information indicating the evaluation result of the protein evaluation of the algae grown in the growing environment specified by the growth ID. The environment evaluation information includes environment color information and environment protein information. The environment color information is information indicating the color of the algae grown in the growing environment. The environment protein information is information indicating the protein content of the algae grown in the growing environment. The environment classification information associated with the growth ID is information indicating the result of the application classification processing of algae grown in the growing environment specified by the growth ID. The environment evaluation information and the environment classification information associated with the growth ID are based on the evaluation result and the application classification processing of the measurement sample specified by the sample ID associated with the growth ID.

The color sample information is information indicating a relationship between algae color and algae protein content. Algae have a property of having different colors depending on protein content. The color sample information stores protein content for each algae color, for example, by associating a color ID with protein content. The color ID is identification information uniquely determined for each color. The color ID may be a number dedicated to the management system 1, or may be a number not dedicated to the management system 1 such as a color code. The color sample information may be created in advance, for example, by measuring protein content of algae of various colors.

The classification information is information indicating a suitable application of the algae for each protein content. Examples of applications of algae include biofuels, bioplastics, feeds, alternative meats, foods, cosmetics, supplements and vaccines.

The classification information indicates, as an example, an intended application of algae whose protein content is equal to or greater than a threshold TH1 as a protein source. In addition, the classification information indicates the intended application of algae whose protein content is less than the threshold TH1 as a starch source. The threshold TH1 may be a predetermined value, for example, 50%. In addition, the classification information indicates that the intended application of algae whose application is a starch source is sugar production, ethyl alcohol production, liquid fuel production, solid fuel production, bioplastic production, and the like.

The communication interface 150 is an interface through which the management apparatus 100 communicates via the network NW or the like.

The input device 160 receives an operation by an operator of the management apparatus 100 (hereinafter, simply referred to as an “operator”). Examples of the input device 160 include a keyboard, a keypad, a touchpad, a mouse, or a controller. The input device 160 may be a device for receiving audio.

The display device 170 displays a screen for notifying the operator or the like of various kinds of information. Examples of the display device 170 include a display such as a liquid crystal display or an organic EL (Electro-luminescence) display. A touch screen can also be used as the input device 160 and the display device 170. That is, a display screen included in the touch screen can be used as the display device 170, and a pointing device using touch input included in the touch screen can be used as the input device 160.

The camera 180 captures an image. The camera 180 outputs captured image data. It should be noted that a moving image is a kind of an image.

The bus 190 includes a control bus, an address bus, a data bus, and the like, and transmits signals transmitted and received by each component of the management apparatus 100.

The color chart 200 is an instrument used for the protein evaluation. As described above, algae have a property of having different colors depending on the protein content. The color chart 200 can utilize this property to measure the protein content of the algae. The color chart 200 includes a plurality of kinds of color samples 201. Different kinds of color samples 201 are colored in different colors. The protein content is determined for each color of the algae. Each color sample 201 is denoted by a corresponding number. The number is the color ID of the color colored in the color sample 201. The color chart 200 shown in FIG. 1 includes twelve kinds of color samples 201 as an example. The 12 kinds of color samples 201 are denoted by numbers 1 to 12, respectively. The numbers 1 to 12 are color IDs.

By comparing the algae color with the color sample 201 and selecting the color sample 201 closest to the algae color, the algae protein content can be measured.

The arrangement of the color sample 201 shown in FIG. 1 is radial, but may be another arrangement such as a matrix. In the color chart 200 shown in FIG. 1, all color samples 201 are integrated. However, the color chart 200 may be divided into a plurality of pieces.

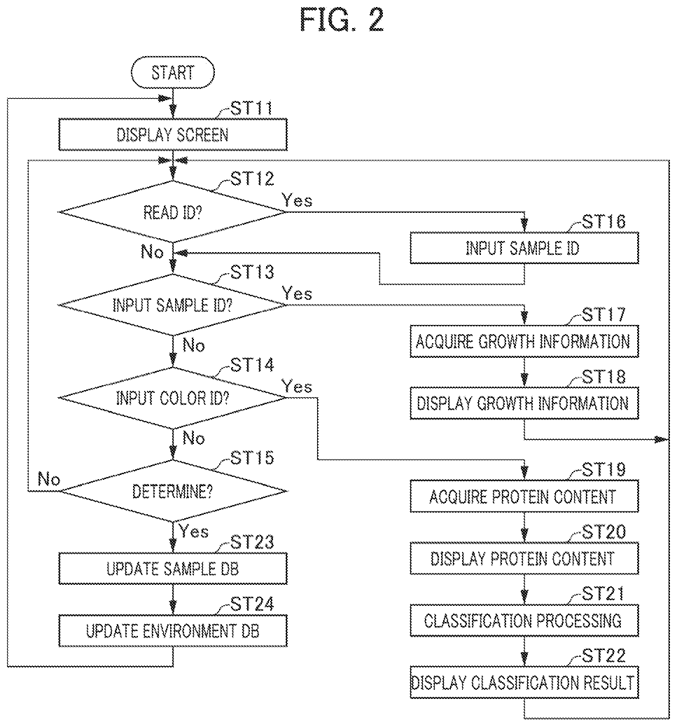

Hereinafter, the operation of the management system 1 according to an embodiment will be described with reference to FIG. 2, etc. It should be noted that the contents of the processing in the following operation description are only examples, and various kinds of processing capable of obtaining similar results can be appropriately used. FIG. 2 is a flowchart showing an example of processing by the processor 110 of the management apparatus 100. The processor 110 executes the processing of FIG. 2 based on a program stored in, for example, the ROM 120 or the auxiliary storage device 140.

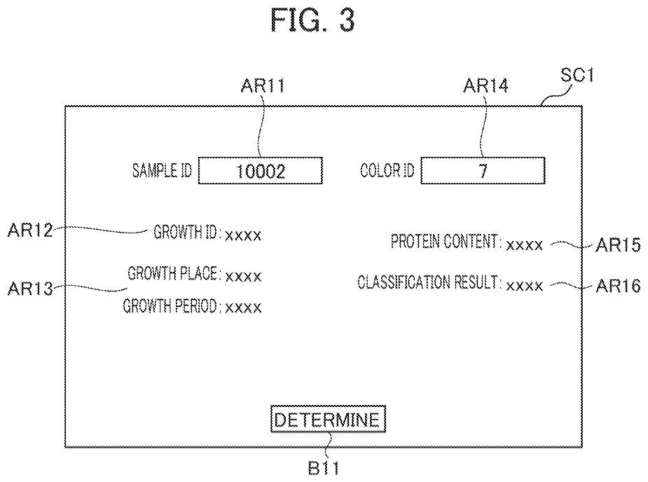

In step ST11 of FIG. 2, the processor 110 of the management apparatus 100 generates an image corresponding to the screen SC1 as shown in FIG. 3. Further, the processor 110 instructs the display device 170 to display the generated image. Upon receiving the instruction for display, the display device 170 displays the screen SC1.

FIG. 3 is a diagram showing an example of a screen SC1 displayed on the display device 170. The screen SC1 is a screen for inputting information necessary for the protein evaluation of the algae measurement sample. Further, the screen SC1 is a screen for displaying growth information of the measurement sample, protein content based on the protein evaluation, and classification results. The screen SC1 includes, for example, areas AR11 to AR15 and a button B11.

The area AR11 is an input field for inputting a sample ID of a measurement sample to be subjected to the protein evaluation.

The area AR12 is an area for displaying the growth ID of the measurement sample specified by the sample ID inputted to the area AR11.

The area AR13 is an area for displaying the growing information specified by the growth ID displayed in the area AR12.

The area AR14 is an input field for inputting a color ID indicating the evaluation result of the measurement sample.

The area AR15 displays the protein content of the measurement sample based on the evaluation result of the protein evaluation.

The area AR16 is an area for displaying the classification result of the measurement samples. The area AR16 displays, for example, an intended application suitable for a measurement sample as a result of the classification. It should be noted that, in the screen SC1 at the time of the processing of step ST11, the areas AR11 to AR16 are typically blank.

The button B11 is operated by the operator when the evaluation result and the classification result of the protein evaluation are determined. The button B11 is operated by the operator when instructing the management apparatus 100 to store evaluation results and classification results.

In step ST12, the processor 110 determines whether or not the sample ID has been read. The sample ID may be manually inputted to the area AR11, or may be read using the camera 180. For example, a container containing a measurement sample displays a sample ID. For example, the container displays the sample ID by being printed the sample ID. The sample ID printed on the container may be a character or a bar code storing the sample ID. The barcode may be a one-dimensional code or a two-dimensional code. The processor 110 acquires an image captured by the camera 180 for determination in step ST12. The processor 110 then attempts to read the sample ID from the image. When the sample ID appears in the image, the processor 110 reads the sample ID by image processing. The processor 110 determines No in step ST12 except for a case in which the sample ID has been read, and then advances to step ST13.

The sample ID is an example of the first identification information. Accordingly, the processor 110, which reads the sample ID, functions as an example of an inputter that receives input of the first identification information. Further, the processor 110, which reads the sample ID, functions as an example of an inputter that receives input of first identification information by reading the first identification information from an image captured by a camera.

In step ST13, the processor 110 determines whether or not the sample ID has been inputted to the area AR11. The processor 110 determines No in step ST13 except for a case in which the sample ID has been inputted to the area AR11, and then advances to step ST14.

In step ST14, the processor 110 determines whether or not the color ID has been inputted to the area AR14. The processor 110 determines No in step ST14 except for a case in which the color ID has been inputted to the area AR14, and advances to step ST15.

The color ID is an example of second identification information. Accordingly, the processor 110 receives the input of the color ID, and functions as an example of a specifying unit that specifies the second identification information given to the color sample closest to the color of the microalgae among a plurality of color samples for comparison with the color of the microalgae.

In step ST15, the processor 110 determines whether or not the button B11 has been operated. In a case in which the button B11 has not been operated, the processor 110 determines No in step ST15 and returns to step ST12. Thus, the processor 110 enters a standby state in which steps ST12 to ST15 are repeated until the sample ID is read, the sample ID is inputted to the area AR11, the color ID is inputted to the area AR14, or the button B11 is operated.

When the sample ID is read in the waiting state in which steps ST12 to ST15 are repeated, the processor 110 determines YES in step ST12 and advances to step ST16.

In step ST16, the processor 110 inputs the read sample ID to the area AR11. After the processing of step ST16, the processor 110 advances to step ST13.

The operator inputs the sample ID of the measurement sample to the area AR11 using the input device 160. As described above, the processor 110 inputs the sample ID to the area AR11 in step ST16.

When the sample ID is inputted to the area AR11 in the standby state in which steps ST12 to ST15 are repeated, the processor 110 determines YES in step ST13, and then advances to step ST17.

As described above, the processor 110, which receives the input of the sample ID using the input device 160, functions as an example of the inputter that receives the input of the first identification information.

In step ST17, the processor 110 acquires the growth information of the measurement sample specified by the sample ID inputted to the area AR11. For this purpose, the processor 110 refers to the sample DB 141 and acquires the growth ID associated with the sample ID. Further, the processor 110 refers to the environment DB 142 and acquires the growth information associated with the growth ID.

In step ST18, the processor 110 controls the display device 170 to display the contents of the growth information acquired in step ST17 in the area AR12. After the processing of step ST18, the processor 110 returns to step ST12.

The operator or the like compares the color of the algae as the measurement sample with the color of the color sample 201 on the color chart 200. Then, the operator or the like inputs the color ID corresponding to the color sample 201 of the color closest to the color of the algae into the area AR14. When the color ID is inputted to the area AR14 in the standby state in which steps ST12 to ST15 are repeated, the processor 110 determines YES in step ST14, and then advances to step ST19.

In step ST19, the processor 110 specifies the protein content of the measurement sample specified by the sample ID inputted to the area AR11, based on the color ID inputted to the area AR14. That is, the processor 110 refers to the color sample information and acquires the protein content associated with the color ID.

In step ST20, the processor 110 controls the display device 170 to display the protein content acquired in step ST19 in the area AR15.

In step ST21, the processor 110 executes application classification processing for the measurement sample specified by the sample ID inputted to the area AR11. That is, the processor 110 refers to the classification information and acquires the intended application suitable for the algae of the protein content acquired in step ST19. For example, when the protein content is greater than or equal to the threshold TH1, the processor 110 determines the intended application as a protein source. On the other hand, when the protein content is less than the threshold TH1, the processor 110 determines the intended application as a starch source.

As described above, the processor 110, which executes the processing of step ST21, functions as an example of the classifier that classifies the microalgae in accordance with the protein content by intended application.

In step ST22, the processor 110 controls the display device 170 to display the result of the application classification processing in step ST21 in the area AR16. After the processing of step ST22, the processor 110 returns to step ST12.

When the button B11 is operated in the standby state in which steps ST12 to ST15 are repeated, the processor 110 determines YES in step ST15 and advances to step ST23.

In step ST23, by updating the sample DB 141, the processor 110 updates the information relating to the measurement sample specified by the sample ID inputted to the area AR11. That is, the processor 110 rewrites the sample color information of the sample evaluation information associated with the sample ID to indicate the color ID inputted to the area AR14. Then, the processor 110 rewrites the sample protein information of the sample evaluation information to indicate the protein content acquired in step ST19. Further, the processor 110 rewrites the sample classification information associated with the sample ID so as to indicate the result of the application classification processing in step ST21. Further, the processor 110 sets the measurement date/time information associated with the sample ID as the current date/time.

As described above, the auxiliary storage device 140 functions as an example of a storage that stores the first identification information and the second identification information to be associated with each other. Further, the processor 110, which executes the processing of step ST23, functions as an example of a storage that stores the first identification information and the second identification information in the storage to be associated with each other.

In step ST24, by updating the environment DB 142, the processor 110 updates the information relating to the growing environment specified by the growth ID acquired in step ST17. That is, the processor 110 rewrites the environment color information of the environment evaluation information associated with the growth ID to indicate the color ID inputted in the area AR14. Then, the processor 110 rewrites the environment protein information of the environment evaluation information to indicate the protein content acquired in step ST19. The protein content indicates the protein content of the algae grown in the growing environment specified by the growth ID. Further, the processor 110 rewrites the environment classification information associated with the growth ID to indicate the result of the classification processing in step ST21. The result indicates an application suitable for algae grown in the growing environment specified by the growth ID.

The processor 110 may determine the evaluation result indicated by the environment evaluation information using the evaluation result of one or more measurement samples having the same growth ID. The processor 110 refers to the sample DB 141 and identifies the sample ID associated with the growth ID acquired in step ST17. The processor 110 may identify the sample ID associated with the growth ID only for the sample ID associated with the measurement date/time information that indicating within a predetermined period.

Then, the processor 110 acquires the protein content from the sample evaluation information associated with each of the identified sample IDs. The processor 110 then obtains a statistical value for one or a plurality of the protein contents acquired. The statistical value is, for example, an average value, a median value, or a mode. The average value may be obtained by using a trim average or a Winsorized mean. Further, the processor 110 may obtain a statistical value after performing a process of excluding outliers for the acquired protein content. Then, the processor 110 rewrites the environment protein information associated with the growth ID acquired in step ST17 to indicate the statistical value. The statistical value indicates the protein content of the algae grown in the growing environment specified by the growth ID.

Further, the processor 110 may perform the application classification processing using the statistical value. That is, the processor 110 refers to the classification information and acquires the application suitable for the algae whose protein content is the statistical value. Then, the processor 110 writes the intended application in the environment classification information associated with the growth ID acquired in step ST17. After the processing of step ST24, the processor 110 returns to step ST11.

According to the management system 1 of the first embodiment, the management apparatus 100 stores the inputted sample ID in association with the color ID of the color sample 201 closest to the color of the measurement sample specified by the sample ID. As described above, the management apparatus 100 according to the first embodiment can reduce the time and effort required to manage the evaluation result of the measurement sample, whereby it is possible to reduce the time and effort required to measure the protein content.

Further, according to the management system 1 of the first embodiment, the management apparatus 100 specifies the protein content from the color ID of the color sample 201 closest to the color of the measurement sample. As described above, since the management apparatus 100 according to the first embodiment automatically specifies the protein content from the color ID, it is possible to reduce the time and effort of measuring the protein content.

Further, according to the management system 1 of the first embodiment, the management apparatus 100 classifies the intended application of algae according to the protein content of algae. As described above, since the management apparatus 100 according to the first embodiment automatically classifies the intended application, it is possible to reduce the time and effort of classifying the intended application.

Further, according to the management system 1 of the first embodiment, the management apparatus 100 classifies the application as a protein source when the protein content of the algae is equal to or greater than the threshold value TH1, and classifies the application as a starch source when the protein content of the algae is less than the threshold value TH1. In this way, the management apparatus 100 of the first embodiment can appropriately classify the intended application of algae.

According to the management system 1 of the first embodiment, the management apparatus 100 reads the sample ID from the image captured by the camera 180. With such a configuration, it is possible for the management apparatus 100 according to the first embodiment to reduce the time and effort of measuring the protein content.

### Second Embodiment

In the management system 1 of the second embodiment, the description of the same portions as in the first embodiment will be omitted.

Since the configuration of the management system 1 of the second embodiment is similar to that of the first embodiment, a description thereof will be omitted.

However, in the management apparatus 100 of the second embodiment, the color chart 200 is installed within the imaging range of the camera 180.

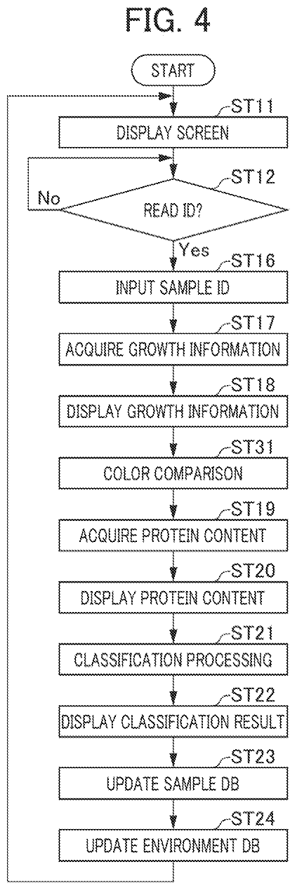

Hereinafter, the operation of the management system 1 according to the embodiment will be described with reference to FIG. 4 and the like. It should be noted that the contents of the processing in the following operation description are only examples, and various kinds of processing capable of obtaining similar results can be appropriately used. FIG. 4 is a flowchart showing an example of processing by the processor 110 of the management apparatus 100. The processor 110 executes the processing of FIG. 4 based on a program stored in, for example, the ROM 120 or the auxiliary storage device 140.

In the second embodiment, a container containing a measurement sample is placed within the imaging range of the camera 180. The container displays a sample ID specifying the measurement sample. The container may be placed in the imaging range of the camera 180 by a person, a robot such as a manipulator, or a conveying device such as a belt conveyor. Further, the processor 110 may move the camera 180 so that the container can enter the imaging range of the camera 180.

In the second embodiment, the processor 110 repeats the processing of step ST12 when the processor 110 determines No in step ST12 of FIG. 4. Therefore, the processor 110 repeats the processing of step ST12 until the sample ID is read.

In the second embodiment, the processor 110 advances to step ST17 after the processing of step ST16. In the second embodiment, the processor 110 advances to step ST31 after the processing of step ST18.

In step ST31, the processor 110 compares the color of the algae, which is a measurement sample, with the color of the color sample 201 of the color chart 200 by image processing of the image captured by the camera 180. Then, the processor 110 identifies the color sample 201 of the color closest to the color of the algae by the image processing. Then, the processor 110 inputs the color ID corresponding to the specified color sample 201 to the area AR14. After the processing of step ST31, the processor 110 advances to step ST19.

As described above, the processor 110 receives the input of the color ID by performing the processing of step ST31, thereby functions as an example of the specifying unit that specifies the second identification information given to the color sample closest to the color of the microalgae among the plurality of color samples for comparison with the color of the microalgae. Further, the processor 110 performs the processing of step ST31, thereby functioning as an example of a specifying unit that specifies the second identification information of the color sample closest to the color of the microalgae among the plurality of color samples by using the image showing the microalgae.

In the second embodiment, the processor 110 advances to step ST23 after the processing of step ST22.

According to the management system 1 of the second embodiment, the management apparatus 100 can obtain the same advantageous effect as that of the first embodiment.

According to the management system 1 of the second embodiment, the management apparatus 100 compares the color of the algae with the color of the color sample 201 of the color chart 200, and specifies the color sample 201 of the color closest to the color of the algae. With such a configuration, it is possible for the management apparatus 100 to automatically perform the protein evaluation.

According to the management system 1 of the second embodiment, the management apparatus 100 is provided with the color chart 200 in the imaging range of the camera 180. With such a configuration, it is possible for the management apparatus 100 according to the second embodiment to reduce error in the protein evaluation due to a change in color of the algae and the color sample 201 due to the influence of light or the like.

The first and second embodiments described above can be modified as follows. In the above embodiments, the classification information is information indicating a suitable application of the algae for each protein content. However, the classification information may indicate the suitable application of the algae for each color ID. In this case, the processor 110 executes the application classification processing using the color ID inputted to the area AR14 instead of the protein content acquired in step ST19.

The management apparatus 100 may include a plurality of apparatuses.

The processor 110 may implement a part or all of the processes implemented by the programs in the above embodiments by a hardware configuration of the circuit.

The program for implementing the processing of the embodiments is transferred in a state of being stored in, for example, a non-transitory storage medium in the apparatus. However, the apparatus may be transferred in a state where the program is not stored. The program may be separately transferred and written to the apparatus. The transfer of the program at this time can be realized, for example, by recording the program in a removable non-transitory storage medium or downloading the program via a network such as the Internet or a local area network (LAN).

While embodiments of the present invention have been described, these embodiments have been presented as example only, and are not intended to limit the scope of the inventions. Embodiments of the present invention can be implemented in various modes without departing from the gist of the present invention.

### EXPLANATION OF REFERENCE NUMERALS

1 management system
100 management apparatus
110 processor
120 ROM
130 RAM
140 auxiliary storage device
141 sample DB
142 environment DB
150 communication interface
160 input device
170 display device
180 camera
190 bus
200 color chart
201 color sample

### ACCESSION NUMBER

FERM BP-22306

What is claimed is:

1. An algae information management apparatus comprising:
an inputter that receives input of first identification information given for each measurement microalgae;
a specifying unit that specifies second identification information given to a color sample closest to a color of the microalgae among a plurality of color samples for comparison with the color of the microalgae; and
storage that stores the first identification information and the second identification information to be associated with each other.

2. The algae information management apparatus according to claim 1, wherein the specifying unit specifies a protein content of the microalgae based on the second identification information.

3. The algae information management apparatus according to claim 2, further comprising a classifier that classifies the microalgae in accordance with the protein content by intended application.

4. The algae information management apparatus according to claim 3, wherein the classifier classifies the intended application as a protein source when the protein content is equal to or greater than a predetermined threshold, and classifies the intended application as a starch source when the protein content is less than the predetermined threshold.

5. The algae information management apparatus according to claim 1, wherein the inputter receives input of the first identification information by reading the first identification information from an image captured by a camera.

6. The algae information management apparatus according to claim 1, wherein the specifying unit specifies the second identification information of the color sample closest to the color of the microalgae among the plurality of color samples by using an image showing the microalgae.

7. The algae information management apparatus according to claim 1, wherein the microalgae is microalgae belonging to Chlamydomonas reinhardtii.

8. The algae information management apparatus according to claim 1, wherein the microalgae is Honda DREAMO strain, accession number: FERM BP-22306.

9. A non-transitory computer-readable storage medium storing a program, the program being executable to cause a processor included in an algae information management apparatus to function as:
an inputter that receives input of first identification information given for each measurement microalgae;
a specifying unit that specifies second identification information given to a color sample closest to a color of the microalgae among a plurality of color samples for comparison with the color of the microalgae; and
a storage controller that stores the first identification information and the second identification information in a storage to be associated with each other.

10. An algae information management method comprising:
receiving input of first identification information given for each measurement microalgae;
specifying second identification information given to a color sample closest to a color of the microalgae among a plurality of color samples for comparison with the color of the microalgae; and
storing the first identification information and the second identification information in association with each other.
